(19) United States
(12) Patent Application Publication
(10) Pub. No.: US 2004/0153168 A1
(43) Pub. Date: Aug. 5, 2004

(54) Shape and roll prosthetic foot

(76) Inventors: Dudley S. Childress, Wilmette, IL (US); Michel Sam, Berkeley, CA (US); Andrew H. Hansen, Round Lake, IL (US); Margrit R. Meier, Chicago, IL (US); Erick H. Knox, Naperville, IL (US)

Correspondence Address: Mr. Edward J. Timmer, P.O. Box 770, Richland, MI, 49083-0770, US

(21) Appl. No.: 10/700,358

(22) Filed: Nov. 3, 2003

Related U.S. Application Data
(60) Provisional application No. 60/424,407, filed on Nov. 6, 2002.

(51) Int. Cl. A61F 2/66

(52) U.S. Cl. 623/55

(57) ABSTRACT

Prosthetic foot is provided having at least one flexible sole element that is deflected under load during a gait cycle of a user of the foot and a plurality of constraining elements that constrain deflection of the at least one flexible sole element such that it assumes an appropriate roll-over shape during a gait cycle of the user. The constraining elements dictate the roll-over shape assumed by the flexible sole element, while the at least one flexible sole element stores and returns energy during the gait cycle of the user to promote a natural “feel” to the foot.

This application claims the benefits of U.S. provisional application Serial No. 60/424,407 filed Nov. 6, 2002.

### FIELD OF THE INVENTION

The invention relates to a prosthetic foot as well as method of making it and, in particular, to a prosthetic foot having a flexible sole element whose deflection under loading is controlled by relatively rigid elements to produce a roll-over shape having an appropriate radius of curvature for the person using the prosthetic foot.

### BACKGROUND OF THE INVENTION

Over the last 15 years there has been a dramatic increase in the number of prosthetic foot designs, and many of them have been classified as “dynamic response” or “energy storing” prosthetic feet. Manufacturers of prosthetic feet and researchers in the field have focused on the characteristic and/or ability of the feet to store and return energy through flexibility of their component parts. The process of deflecting the foot under the loads of walking stores energy within the foot structure, which is then released when the foot is undeflected. Users tend to prefer these foot types, but scientific bases for their preference have not been forthcoming. Studies show that the act of deflecting these commercially available prosthetic feet does indeed store energy (as do all feet).

A prosthetic foot having a so-called roll-over shape whose profile is important to walking is described by E. Knox, in The Role of Prosthetic Feet in Walking. Ph.D. Thesis, Northwestern University, Evanston, Ill., 1996, and cataloged as Diss 378 NU 1996 K74r in the library of Northwestern University. That prosthetic foot consisted of a wooden rocker keel with a polypropylene cantilever sole plate attached to the bottom of the rocker keel. That prosthetic foot provides the user with the desired roll-over shape and energy return.

The present invention provides a prosthetic foot that improves on the prosthetic foot of the preceding paragraph.

### SUMMARY OF THE INVENTION

The invention provides in one embodiment a prosthetic foot comprising at least one flexible element that is deflected under load during a gait cycle of a user of the foot and a plurality of constraining elements that constrain deflection of the at least one flexible element such that it assumes an appropriate roll-over shape during a gait cycle of the user. The constraining elements dictate the roll-over shape assumed by the flexible element, while the at least one flexible element stores and returns energy during the gait cycle of the user to promote a natural “feel” to the foot.

In a preferred embodiment of the invention, the flexible element typically comprises the sole of the prosthetic foot and the constraining elements are disposed on the flexible element and interconnected by regions of the flexible element. The constraining elements are spaced apart by upstanding gaps in a direction of the foot longitudinal axis and are adapted to abut one another in a manner to constrain maximum deflection of the flexible element such that it forms an approximate circular arc roll-over shape during a gait cycle of the user. The prosthetic foot provides a smooth, consistent, and comfortable walking gait by providing the user with an appropriate ankle-foot roll-over shape (or ankle-foot-shoe roll-over shape if a shoe is used with the foot). The ankle-foot roll-over shape is defined as the effective geometry, or rocker, to which the ankle-foot system conforms between heel contact and opposite heel contact events during walking. The prosthetic feet can be designed to give a specific desired roll-over geometry for walking. The prosthetic foot permits the stiffness of the foot and the roll-over shape of the foot to be essentially uncoupled; i.e. one can be changed without affecting the other. In contrast, in previous commercially available prosthetic feet, the stiffness of the foot is coupled with the roll-over shape of the foot such that changing the stiffness will change the roll-over shape, and vice versa.

Moreover, the prosthetic foot of the invention can fit into a shoe of the user, thereby thus promoting clinical use of the foot by patients. The prosthetic foot can be made using readily available light-weight materials by hand or machine manufacture at low cost.

The above and other advantages of the invention will become more readily apparent from the following description taken with the following drawings.

### DESCRIPTION OF THE DRAWINGS

FIG. 1A is a side elevation of a prosthetic foot pursuant to an illustrative embodiment of the invention.

FIG. 1B is a side elevation of a prosthetic foot pursuant to another illustrative embodiment of the invention.

FIG. 1C is a side elevation of a prosthetic foot pursuant to another illustrative embodiment of the invention.

FIG. 2 is a plan view of the prosthetic foot of FIG. 1A.

FIG. 3A is a side elevation of the prosthetic foot showing an insert therein.

FIG. 3B is a sectional view of the prosthetic foot of FIG. 3A taken along lines 3B.

FIG. 4 is a side elevation view of the prosthetic foot of FIG. 1C connected to a pylon connector of a residual limb socket.

FIG. 5 is a schematic view of the deflection of the prosthetic foot at its maximally deflected position and constrained at a desired radius of curvature.

FIG. 6 is an exploded view of a compression mold for making the prosthetic foot.

### DETAILED DESCRIPTION OF THE INVENTION

The invention provides a prosthetic foot for use by a person with lower limb amputation in a manner to gradually transfer walking load from the heel to the toe. During this period of time, the stance knee traverses a somewhat level path over the foot in the direction of walking. The precise location of the knee joint with respect to the ground contact point is influenced primarily by the deformation of the materials of the prosthetic foot and shoe, as well as the tissues of the residual limb. The dynamic actions that are occurring within this time period effectively create a consistent relationship between the knee and ground contact point such that an appropriately prosthetic foot can produce the same consistent knee-to-ground relationship. The prosthetic foot provides an appropriate roll-over shape as defined above and below to enable a person with lower limb amputation to walk comfortably and smoothly and also provides shock attenuation and energy storage/return to improve the “feel” of walking. The prosthetic foot embodies the dynamics of deflecting the foot under the loads of walking to store energy within the foot structure, which is then released when the foot is undeflected.

The prosthetic foot achieves smooth, consistent, and comfortable walking by providing the user an appropriate ankle-foot roll-over shape (or ankle-foot-shoe roll-over shape). The ankle-foot roll-over shape is defined as the effective geometry, or rocker, to which the ankle-foot system conforms between heel contact and opposite heel contact events during walking. The prosthetic foot pursuant to the invention provides an appropriate roll-over shape to give a specific desired geometry for walking.

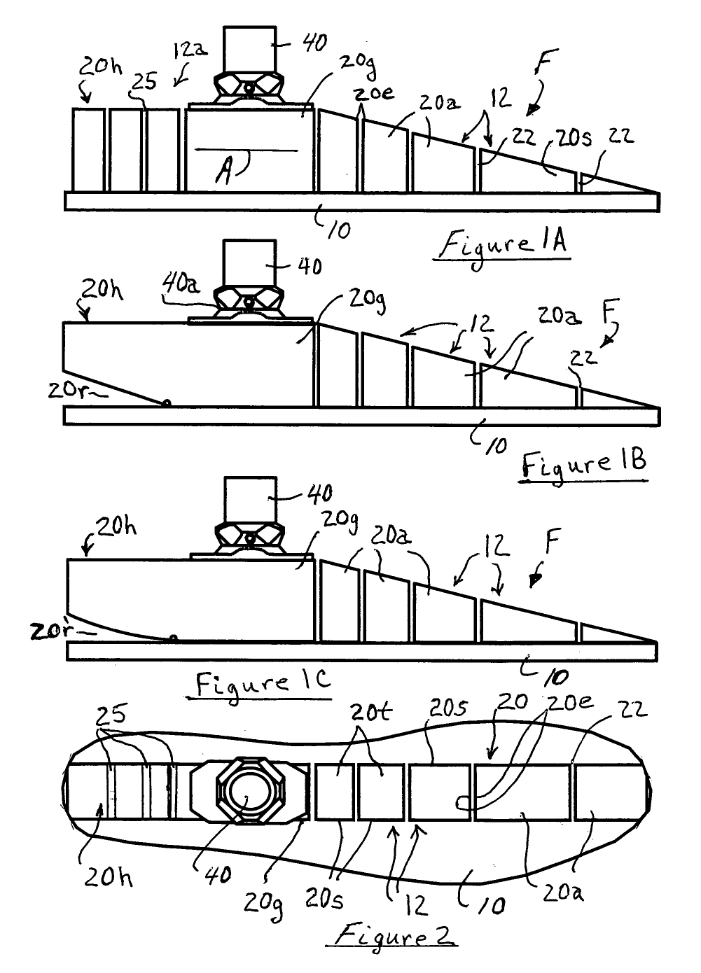

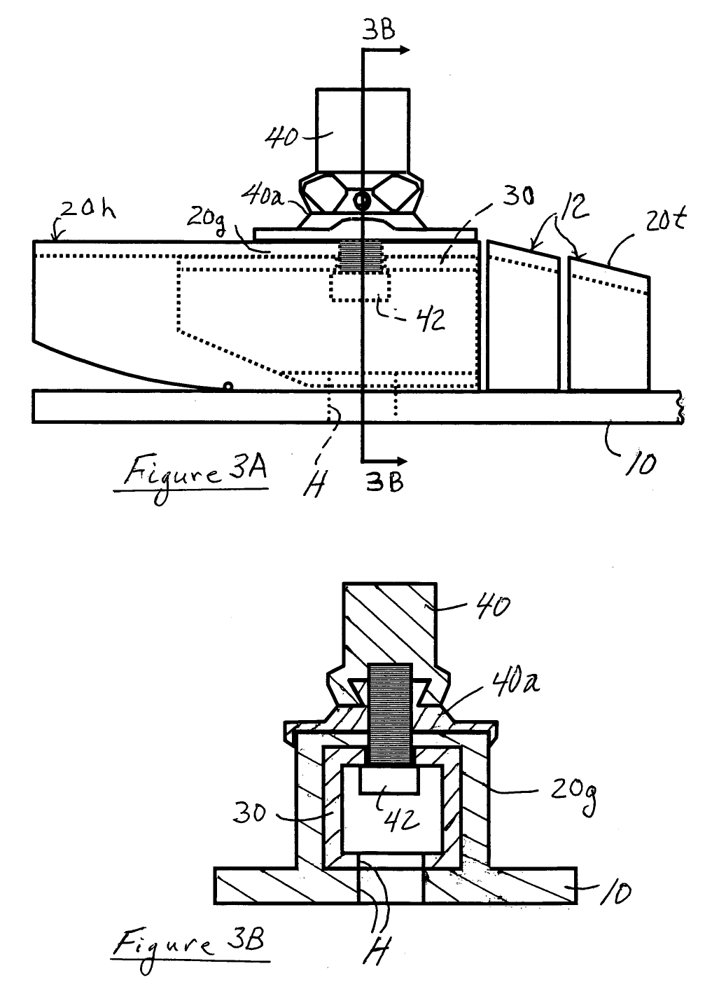

The invention provides a prosthetic foot comprising at least one flexible element that is deflected under load during a gait cycle of a user of the foot and a plurality of constraining elements that constrain deflection of the at least one flexible element such that it assumes an appropriate roll-over shape during a gait cycle of the user. The constraining elements dictate the roll-over shape assumed by the flexible element, while the at least one flexible element stores and returns energy during the gait cycle of the user to promote a natural “feel”, to the foot. Although the combination of one or more flexible elements and constraining elements pursuant to the invention can be implemented in a variety of ways, certain illustrative embodiments will be described below with respect to FIGS. 1-6.

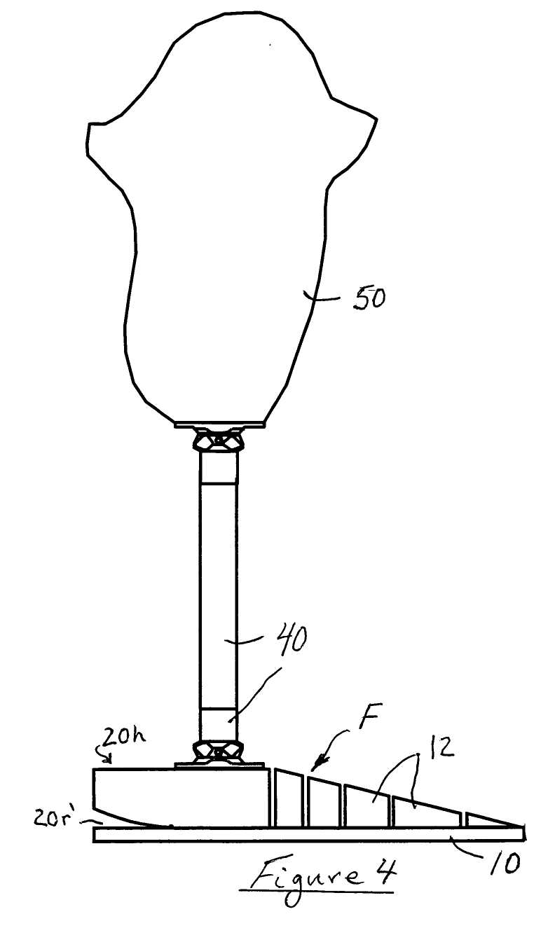

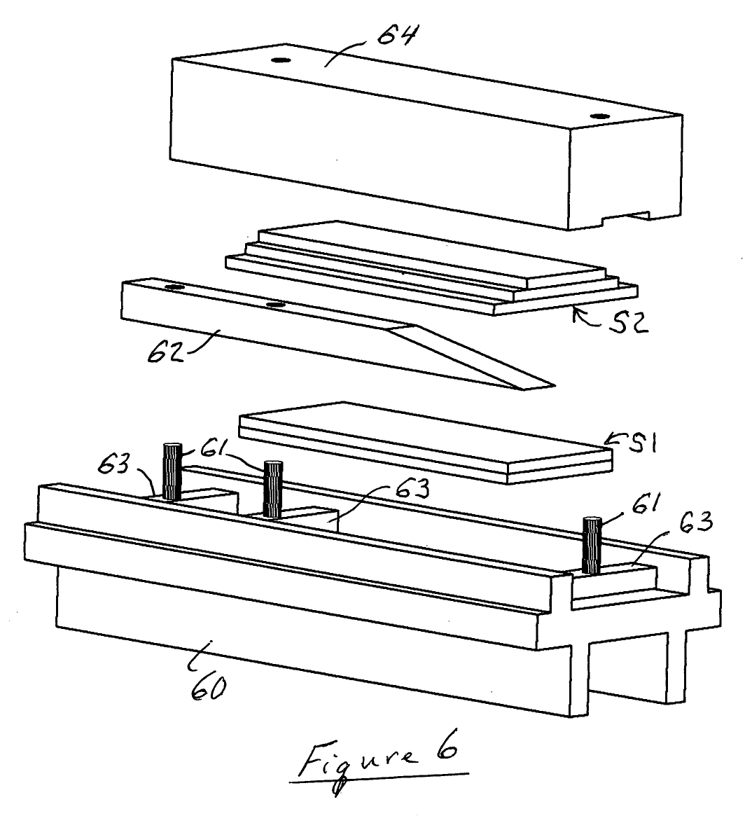

Referring to FIGS. 1A and 2, an illustrative embodiment of the invention is shown to comprise at least one flexible element 10 that is shown comprising or forming the flexible sole element of the prosthetic foot F and a plurality of the constraining elements 12 disposed on the flexible element 10 and spaced apart by upstanding gaps 22 in a direction of the foot longitudinal axis A for constraining maximum deflection of the flexible element 10 in a manner to form an approximate circular arc roll-over shape, as shown in FIG. 5 for purposes of illustration, during a gait cycle of the user. The flexible element 10 and constraining elements 12 can be made as one-piece integrally joined to one another or, alternately, they can be made as separate foot components that are connected together in any suitable manner to achieve appropriate functioning of the prosthetic foot pursuant to the invention.

In the prosthetic foot F shown, the flexible sole element 10 is designed to deflect or bend until the constraining elements 12 contact each other as described below to prevent further deflection or bending of the flexible element. That is, the constraining elements 12 function as substantially incompressible rigid elements after they contact one another to prevent further deflection or bending of the flexible element. In FIG. 1A, the flexible element 10 comprises, in the undeflected or unbent condition, a flat sole plate member having in a plan view a profile or outline generally of a human foot, FIG. 2. The flexible sole element 10 is adapted to deflect or bend in an arcuate shape during the gait of the user of the foot until the constraining elements 12 act to prevent further any deflection or bending of the flexible element beyond a maximum deflected position. An illustrative maximum deflected condition of the flexible element 10 as constrained by the constraining elements 12 is shown in FIG. 5 where the flexible element 10 has assumed an approximate circular arc shape at maximum deflection or bending thereof.

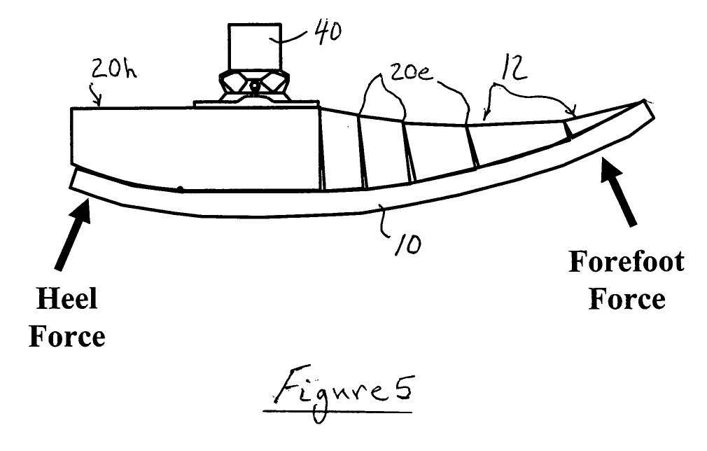

The constraining elements 12 are shown as having a trapedoizal bar shape when viewed in elevation from the side of the foot, FIG. 1A, and a rectangular cross section in FIG. 3B, although any suitable configuration and geometry or combination thereof can be used as long as their interaction results in constraining the flexible element 10 to the appropriate roll-over shape at the maximum deflected condition. The constraining elements 12 are formed in an illustrative embodiment of the invention by a hollow tapered elongated bar 20 molded on the flexible element 10 as described below and into which saw-cuts or gaps 22 are made through the top wall 20t and the side walls 20s of the bar 20 at predetermined intervals along the length thereof to provide a plurality of bar segments 20a separated from one another by upstanding gaps 22, leaving the bottom of the bar 20 uncut. Before saw-cutting, the bottom of the bar 20 is closed and formed by that region of the flexible element 10 residing below the top side 20t. As a result, after saw-cutting, the flexible element 10 may be thought as including a plurality of flexible regions 10a that interconnect the adjacent bar segments 20a along a length of the foot. The molded and cut bar segments 20a do not need to be tapered, but tapering facilitates fitting of the finished prosthetic foot F into a shoe. Moreover, the invention is not limited to forming the bar segments 20a by saw-cutting of the original molded bar 20 since they can be formed in any suitable manner to serve their intended function of dictating the maximum deflected position of the flexible element 10.

When a load is applied to the prosthetic foot F during a gait cycle of the user, the flexible sole element 10 deflects into an arcuate shape with the maximum deflection limited by the oppositely facing, top transverse edges 20e of the bar segments 20a moving a distance roughly equal to the width of the saw cuts or gaps 22. When the top transverse edges 20e of adjacent bar segments 20a abut respective top edges 20e of adjacent bar segments 20a, the flexible sole element 10 and the tapered bar segments 20a collectively have assumed an approximate circular roll-over shape with a radius of curvature, see FIG. 5. The bar segments 20a become rigid and incompressible when their top transverse edges 20e abut against each other and will not deform much more under extra loads as a result of their shape selected to this end. For example, in FIG. 5, if the Forefoot Force is increased further, the flexible element 10 prosthetic foot F will not deflect further as a result of the abutment of the top transverse edges 20e. The number of saw cuts or gaps 22 and the distance between the sawcuts or gaps are calculated based upon the desired radius of curvature, the height of the rectangular bar 20, and the width of the saw-cuts or gaps 22.

An attachment segment 20g of the rectangular bar 20 is left uncut so that a tight-fitting tubing insert 30 made of a rigid material, such as aluminum, can be pressed into this segment 20g as shown in FIGS. 3A, 3B. This attachment segment 20g provides an uncut, crush-proof attachment region for connection to the pylon connector 40 connected to the residual limb socket 50 of the prosthetic device of FIG. 4.

In particular, an attachment bolt 42 is used to fasten the attachment segment 20g and the upper wall of the insert 30 to the pylon connector flange 40a, FIG. 3. The pylon connector 40 is conventional pylon connector having a threaded connector flange 40a that accepts the attachment bolt 42. The shape of connector flange 40a prevents rotation of the foot.

The flexible sole element 10 comprises the main flexible element of the foot and is the principal element that stores and releases energy during the gait cycle of the user. It also provides shock attenuation at heel contact. Providing a shock-absorbing heel can be achieved in a number of ways. For example, referring to FIG. 1A, saw-cuts or gaps 25 can be provided in the remaining heel segment 20h of the bar 20 to facilitate bending at the heel of the prosthetic foot F. Referring to FIGS. 1B and 1C, the heel segment 20h of the bar 20 can be left uncut from the top wall or surface and a straight wedge-shaped recess 20r, FIG. 1B, or curved wedge-shaped recess 20r′, FIG. 1C, can be cut out from between the flexible sole element 10 and the rectangular bar segment 20h forming the heel of the foot F to provide shock absorption at the heel.

It is apparent that during the roll-over phase of the gait cycle, the flexible sole element 10 is deflected and the top transverse edges 20e of the trapezoidal bar segments 20a of the forefoot come into contact. This contact sets the desired radius of curvature. Then, near the end of the stance phase of the gait cycle, when the loads are rapidly being transferred to the other limb of the user, the flexible sole element 10 undeflects as a result of elastic properties of the material thereof and provides a measure of energy return. Energy also returns from the rectangular bar segments 20a, which functions similar to a foot keel.

The thickness of the flexible sole element 10 is chosen so that the top transverse edges 20e of the trapezoidal bar segments 20a will come into contact under the normal loads of walking. The thickness can be altered to fit the weight and activity level of the user. The flexibility and energy storing properties of the foot can be altered without changing the roll-over shape of the foot. This is an advantageous and unique feature of the prosthetic foot F.

The approximate circular roll-over shape that the prosthetic foot F assumes is chosen to provide smooth consistent progression of the center of pressure under the foot. The rectangular bar segments (foot keel) 20a collectively extend near the end of the foot to provide progression of the center of pressure to the end of the foot under high loads. Selection of the radius of curvature and the location of the center of curvature in the sagittal plane are chosen individually for each user to maximize biomechanical effects during use. Preferably, the radius of curvature is chosen to provide a predominantly horizontal pathway for a point below the knee, and to promote symmetry in the vertical trunk movements between the prosthetic and sound side steps.

Selection of the radius of curvature can be based solely on the person's leg length. Theoretical and experimental determinations have agreed that the radius of curvature be chosen as about 30-40% of the person's leg length. This provides a simple method for providing the proper prosthetic foot F for each individual user. Note that the radius of curvature determination is independent of bodyweight. The length of the prosthetic foot F is matched to the sound side foot of the user. The prosthetic foot F can be used with a cosmetic shell (not shown) for appearance purposes.

The prosthetic foot F can be made of a wide variety of materials. For purposes of illustration and not limitation, entirely out of a polypropylene-polyethylene copolymer, because this material is inexpensive, readily available, waterproof and is known for good mechanical properties. There are numerous ways to manufacture a prosthetic foot F. A simple way may be compression molding.

Referring to FIG. 6, a compression molding fabrication method uses a three-piece mold having a bottom mold piece 60 (made of wood or aluminum) with threaded rods 61 and bars 63 that serve to guide and hold the other two mold pieces in place relative thereto. The middle mold piece 62 is designed to equal the desired hollow part of the rectangular sloped bar 20. The top mold piece 64 is a wide block with a shape that conforms to the top of the middle mold piece 62, leaving space equal to the required thickness of the rectangular bar 20 to be formed therebetween. The top mold piece 64 serves as a negative mold for the top surface of the prosthetic foot. When making a foot, two or more melted, layered sheets S1 of the above copolymer of the appropriate size are laid onto the bottom mold piece 60. Then, the middle mold piece 62 is set at the desired height and held in place by the threaded rods 61. Additional melted, layered sheets S2 of the copolymer are laid on top of the assembly and then the top mold piece 64 is quickly and tightly bolted to the rest of the assembly using rods 61. The thickness of the flexible sole element 10 of the prosthetic foot can be varied by changing the height that the top and middle mold pieces 64, 62 are allowed to compress the plastic sheets S1. This can be achieved by changing the thickness of the bars 63. Additional plastic sheeting is needed in the mold to produce thicker flexible sole elements. The whole apparatus is then allowed to cool to room temperature. The mold is then disassembled, and the middle mold piece 62 is removed from the foot, leaving the desired hollow geometry of the rectangular tapered hollow bar 20, which is subsequently saw-cut as described above.

After the prosthetic foot is made, the uncut attachment segment 20g of bar 20 is mated with the rigid tubing insert 30 (aluminum or other hard material) which is received in the attachment segment. Then, the appropriate saw-cuts 22 are made into the hollow bar 20 to produce the bar segments 20a. Finally the flexible sole element 10 is cut into the profile in plan of a foot sole and rough edges are smoothed. The prosthetic foot is attached to the pylon connector 40 using bolt 42 inserted and tightened via holes H in the flexible sole element 10 and insert 30.

This fabrication method is designed for practice in low-income, undeveloped countries, so that local labor and indigenous materials can he used to make the prosthetic foot. Other fabrication methods better suited for manufacture in the US or other industrialized countries may include extrusion or injection molding or any other suitable manufacturing method.

Although certain embodiments of the invention including at least one flexible element and a plurality of constraining elements have been described above, the invention is not so limited and can be practiced in other ways so long as one or more flexible elements and constraining elements are combined to provide the appropriate roll-over shape after deflection of the flexible element(s) as controlled by the constraining elements. For example, one or more flexible elements may be used together with inextensible elements, such as cords or braids, so that the flexible elements(s) can deflect or bend to a maximum deflected position when the inextensible elements then function to prevent further deflection or bending of the flexible elements.

Moreover, those skilled in the art will appreciate that modifications and changes can be made in the invention without departing from the scope of the invention as set forth in the appended claims.

1. Prosthetic foot, comprising at least one flexible element that is deflected under load during a gait cycle of a user of the foot and a plurality of constraining elements that constrain deflection of the at least one flexible element such that it assumes an arcuate roll-over shape during the gait cycle of the user.

2. The foot of claim 1 wherein the constraining elements are incompressible.

3. The foot of claim 1 wherein the constraining elements are inextensible.

4. The foot of claim 1 wherein the flexible element comprises a flexible sole element of the foot.

5. The foot of claim 4 wherein the constraining elements are disposed on the flexible sole element to constrain deflection thereof so that the flexible sole element assumes the roll-over shape at a maximum deflected position thereof.

6. The foot of claim 5 wherein regions of the flexible sole element reside between and interconnect adjacent constraining elements.

7. The foot of claim 5 wherein the constraining elements are spaced apart by upstanding gaps in a direction of a foot longitudinal axis and are adapted to abut one another in a manner to constrain maximum deflection of the flexible sole element so that it assume the roll-over shape.

8. The foot of claim 7 wherein the roll-over shape is an approximate circular arc roll-over shape.

9. Prosthetic foot, comprising at least one flexible sole element that is deflected under load during a gait cycle of a user of the foot and a plurality of constraining elements that are abuttable against one another at a maximum deflected position of the flexible sole element to constrain the at least one flexible sole element to assume an approximate circular arc roll-over shape during the gait cycle of the user.

10. The foot of claim 9 wherein the constraining elements are incompressible.

11. The foot of claim 9 wherein regions of the flexible sole element reside integrally between and interconnect adjacent constraining elements.

12. The foot of claim 5 wherein the constraining elements are hollow bar segments that are spaced apart from one another by upstanding gaps in a direction of a foot longitudinal axis, the bar segments having oppositely facing top edges that are adapted to abut respective top edges of an adjacent bar segment to constrain maximum deflection of the flexible sole element.

13. The foot of claim 9 wherein the constraining elements are formed integrally on the flexible sole element and collectively form a keel of the foot.

14. The foot of claim 9 further including an attachment segment for connection to a pylon connector of a residual limb socket.

15. The foot of claim 9 further including a heel segment that is configured to allow bending of the foot at the heel segment.

16. A method of making a prosthetic foot, comprising connecting at least one flexible element that is deflected under load during a gait cycle of a user of the foot and a plurality of constraining elements that constrain deflection of the at least one flexible element such that the flexible element assumes an arcuate roll-over shape during the gait cycle of the user.

17. The method of claim 16 wherein the at least one flexible element is molded integrally with the constraining elements.

18. The method of claim 16 including forming gaps between adjacent constraining elements in a direction of a foot longitudinal axis.
